(19) United States
(12) Patent Application Publication
(10) Pub. No.: US 2007/0009590 A1
(43) Pub. Date: Jan. 11, 2007

(54) Physiologically active composition based on phosphatidylserine

(76) Inventors: Martin Purpura, Bonn (DE); Ralf Jager, Freising (DE); Roland Rabeler, Munchen (DE); Hans-Ullrich Hoppe, Langenbach (DE)

Correspondence Address: FULBRIGHT & JAWORSKI, LLP, 666 FIFTH AVE, NEW YORK, NY, 10103-3198, US

(21) Appl. No.: 10/570,071

(22) PCT Filed: Sep. 3, 2004
(86) PCT No.: PCT/EP04/09862

§ 371 Date: Mar. 1, 2006

(30) Foreign Application Priority Data
Sep. 4, 2003 (DE) 10340740.5

(51) Int. Cl. A61K 31/685; A61K 9/20

(52) U.S. Cl. 424/464; 514/78

(57) ABSTRACT

This physiologically active composition contains, as physiologically active constituents, the components a) phosphatidylserine and/or lysophosphatidylserine (in each case “PS”) and b) at least one (lyso)phospholipid different from component a) and c) a serine source different from component a), for example L-serine and/or O-phospho-L-serine. In this composition, the components a) and also b) and c) must each be present in a molar ratio a:b (where b<c) and a:c (where c<b) such that the components b) and c) can preferably form PS in vivo and in this manner ideally replace 10 to 99% by weight of the PS portion in the total formulation. The component a) should here make up 0.1 to 20% by weight of the total formulation and the components b) and c) should be present in portions between 15 and 65% by weight and in portions between 0.1 and 5.0% by weight, respectively. This composition which, in addition to the main components, can also contain further, in particular physiologically active constituents, such as omega-3 fatty acids, is used as liquid formulation or else as powder for producing an agent for improving and enhancing and memory function, or as dietary supplement, functional food or special nutrient.

The present invention relates to a physiologically active composition based on phosphatidylserine.

Phosphatidylserine (PS) is a natural phospholipid having typical amphiphilic properties. Together with other representatives of the phospholipids, it takes part in the structure of biological membranes. The corresponding lysoforms do not bear a fatty acid in the C₁ or C₂ position of the glycerol radical, but a single hydroxyl group.

Owing to their chemical properties, phospholipids in general and phosphatidylserine and also its lyso variant in particular take part in the structure of the brain, in general metabolic processes, and in the transmission and processing of neuronal information.

The beneficial effect of phosphatidylserine on the brain function, when it is administered externally, is also adequately known.

Thus, for example U.S. Pat. No. 5,900,409 protects a composition for improving the brain function, which composition contains phosphatidylserine which has been produced by a transphosphatidylation by using phospholipase D. U.S. Pat. No. 6,117,853 describes a corresponding composition which contains lysophosphatidylserine as main active substance.

If, in the past, phosphatidylserine has been solely isolated from brain material, for example, from beef and pigs, in the last decade, the production of PS in commercial amounts by an enzymatic transphosphatidylation in the presence of phospholipase D has established itself.

Not only can phosphatidylserine and/or lysophosphatidylserine have a beneficial effect on the brain function, but also numerous other compounds, for example omega-3 and omega-6 fatty acids, which are likewise essential constituents of neuronal tissue, for example in the brain.

EP-A 0 342 795, for example, also discloses a composition for improving the brain function, which composition contains docosahexaenoic acid (DHA), that is a typical omega-3 fatty acid.

Omega-3 and omega-6 fatty acids, however, are not only used as single preparations, but, on account of their major source, the fish oils, are mostly used in combined preparations, in which case they are then administered in encapsulated form.

Fish oils and their main ingredients, the fatty acids, are used in various ways and are said to have beneficial effects in inflammatory disorders, for example of joints, in improvement of sporting performance, but also for decreasing the serum cholesterol level. In this context, reference is made by way of example to GB 2209936, which teaches a fish oil preparation for treating arthritic symptoms, or else to WO 93/21912, which likewise advises the use of an emulsion containing omega-3 fatty acids for treating inflammatory disorders. Likewise for treating arthritis, according to U.S. Pat. No. 5,843,919, use is made of a combination of omega-3 fatty acids and glucosamine. The treatment of joint disorders, of joint pain and stiffness of joints using rose hip concentrates and fish oils is protected by U.S. Pat. No. 6,485,752. According to EP-A 1155620, a combination of different vitamins with mineral substances and at least 40% by weight of fish oil granules in the form of a dietary supplement is claimed. EP-A 1004303 proposes a composition which contains highly unsaturated omega-3 fatty acids, in order to decrease risk factors during sporting exercise.

In general, in the case of orally administered compositions, there is the problem that the formulations, and in particular their physiologically active ingredients, survive the gastrointestinal tract in such a manner that they reach their intended site of action in active form. This requires that they survive the different pH environments of the gastrointestinal tract and also the enzymatic processes proceeding therein in a manner such that their physiological activity is not adversely changed.

For this purpose, most active substances must be supplied orally in high-dose form, or be formulated in such a manner that a sufficient amount of the unchanged compound passes to the site of action. In the case of oral administration of phosphatidylserine, for example, a series of processes lead to a massive reduction of the final concentration at the site of action. Thus a large portion of the phosphatidylserine taken up in the gastrointestinal tract is already irreversibly converted into other phospholipids in the intestinal cells. At the same time, specific enzymatic degradation processes lead to a further reduction of the effective amount of phosphatidylserine.

On account of this known disadvantage of the prior art, for the present invention the object has been set to provide a physiologically active composition based on phosphatidylserine with which, in particular in the case of oral administration, sufficiently high amounts of active substances of phosphatidylserine or its lysoform are achieved at the different possible sites of action in the body without complex formulations or special administration forms needing to be applied.

This object was achieved by a corresponding composition which contains, as physiologically active constituents, the components
a) phosphatidylserine and/or lysophosphatidylserine (in each case “PS”) and
b) at least one (lyso)phospholipid different from component a) and
c) a serine source different from component a), for example L-serine and/or O-phospho-L-serine,
   the components a) and also b) and c) being present in each case in a molar ratio of a:b=1.0:0.1 to 1000, wherein b<c, and of a:c=1.0:0.1 to 1000, wherein c <b.

Surprisingly, it has been found using this composition that the amounts of components b) and c) are suitable under physiological conditions to increase significantly the concentration of phosphatidylserine at the site of action (in particular brain). In this case, firstly the components b) and c) in the body are converted to phosphatidylserine, secondly their presence compensates for the PS loss due to degradation and conversion, but also improves PS transport to the site of action. This takes place to an extent that the two components b) and c) are not only able to compensate for the amounts of phosphatidylserine-customarily administered and/or catabolized under physiological conditions, but together with the amounts of phosphatidylserine remaining unchanged in activity during passage through the body lead to an amount of total PS at the respective site of action which makes possible a great reduction of phosphatidylserine used. This surprising effect not only has economic consequences, but specific prior calculation of the amount of PS arriving at the principal site of action, the brain, which, in particular, is of importance with respect to the beneficial effect on symptoms of distress in the case of physical and/or mental stress, could also become possible.

An inventive composition has been found to be particularly effective, the molar ratios of which composition a:b and a:c are 1.0:0.5 to 500, and particularly preferably 1.0:1.0 to 100.

However, in the context of the present invention, a variant of the claimed composition has been found to be particularly suitable in which, under in vivo conditions, PS can be formed from the components b) and c), more precisely, preferably in amounts replacing 10 to 99% by weight of the portion of component a), that is phosphatidylserine and/or lysophosphatidylserine, of the total formulation.

In this context, an inventive composition is also considered as preferred which contains 0.1 to 20% by weight, based on the total formulation, of component a). This variant illustrates the potential associated with the inventive composition for decreasing externally administered PS quantities.

Since the main advantage of the claimed composition is therefore that phosphatidylserine and/or lyso-phosphatidylserine portions can be greatly decreased by the combined administration with the components b) and c), the present invention also covers a composition in which component b) is present in portions between 15 and 65% by weight, and the serine component c) is present in portions between 0.1 and 5.0% by weight, again in each case based on the total formulation.

Typical representatives of component b) are phosphatidylcholine (PC), phosphatidylethanolamine (PE) and phosphatidylinositol (PI) which obviously can also be used as lyso variants in the claimed composition; but also other suitable (lyso)phospholipids come into consideration as component b).

With respect to the further main component c), that is the serine source different from PS, the present composition is subject to no restriction in the context of the claimed requirements. It is only necessary to ensure that under physiological conditions serine is actually available for the desired purpose.

In addition to the three components a, b and c essential to the invention, the claimed composition can also contain further physiologically active constituents, in which case preferably additionally omega-3 fatty acids come into consideration. In this context, the present invention provides a portion which is between 30 and 80% by weight, based on the total formulation. This creates the possibility of firstly supporting the physiological PS activity, or else intensifying it synergistically. However, it is also advantageous that this additional component not only serves as active substance, but equally well also as matrix for the three main components.

The use of docosahexaenoic acid (DHA), α-linolenic acid (ALA) and eicosapentaenoic acid (EPA), in particular in the form of mono-, di- and/or triglycerides and monoesters, or else in the form of any desired mixtures as typical representatives of the additional fatty acid component, has proved particularly advantageous.

As mentioned above, the claimed composition, in addition to the main ingredients a) to c) and the omega-3 fatty acids, can also contain further components which either themselves exhibit a physiological activity or are only used as formulation aid. Preference here as further constituents, and in particular in the form of additional physiologically active constituents, is given to those having circulation-promoting activity, e.g. Ginko biloba, or those having neuroprotective and/or antioxidant activity, e.g. vitamin A, vitamin C, vitamin E, polyphenols, beta-carotene, selenium, and α-lipoic acid, but also those constituents having activity stimulating brain metabolism, e.g. complex vitamins (B₁, B₆, B₁₂, folic acid), omega-6 fatty acids, creatine, caffeine and carbohydrates (glucose). Preferably, however, substances can also be comprised which mostly affect the synthesis or release of neurotransmitters, e.g. choline and also (in)organic salts thereof or acetyl donors, e.g. acetylcholine, and/or substances which beneficially affect the bioavailability, distribution and metabolism of phospholipids, e.g. PPAR agonists (that is peroxisome-proliferator-activated-receptor agonists which mostly, thiazolidindione-based, act in an insulin-sensitizing and lipid-lowering manner in treatment of diabetes type II), retinoic acid receptor agonists and blood cholesterol-decreasing compounds in general. Finally, as further constituents, formulation aids come into consideration, e.g. fillers, release agents, flavorings and colors, all said further constituents obviously being able to be present in any desired mixture in the claimed composition.

In addition to the components of the inventive composition, the present invention also comprises their administration form, liquid formulations, and in particular those in capsule form or as powder and in particular as tablet or dragee, being considered as preferred. If capsules are used, these, as carrier matrix, usually contain fish oils; powders consist of or contain microencapsulated fish oils which can also be omega-3 fatty acids.

The present invention also covers the use of the inventive composition for producing an agent for improving and enhancing the brain and memory function, and also their further preferred use as dietary supplements, functional food or as special nutrient.

A further aspect of the inventive use is associated with physical and mental distress, as can occur, in particular, in the context of sporting activities, and which can be prevented by the inventive composition, or whose symptoms are greatly reduced with supplementation.

In summary, the inventive, physiologically active composition is a novel agent by which it is possible to beneficially affect the bioavailability, distribution and metabolism of phospholipids in the body. Phosphatidylserine or its lysoform can in this case be produced for the most part by means of physiological in vivo processes from the physiological precursor substances b) and c) offered, as a result of which the amount of PS (component a)) actually supplied orally can be greatly decreased. The orally supplied (lyso)phosphatidyl amount is in addition supported in its activity at the target site by additional amounts of PS being formed in vivo from the components b) and c), which together with the (lyso)phosphatidylserine (component a)) administered and additional components, such as omega-3 fatty acids, lead to improved function and performance at the main site of action, that is to say the brain.

The examples hereinafter illustrate the advantages of the present invention.

### EXAMPLES

### Example 1

10 g of phosphatidylserine (Leci PS® 90PN from Degussa Food Ingredients GmbH) were added as component a) to 180 g of the omega-3 fatty acid docosahexaenoic acid (Marinol™ D-50 TG from Loders Crooklan) and stirred in a laboratory mixer until a homogeneous mixture resulted. Subsequently, as component b), 180 g of phosphatidylcholine (Epikuron™ 135F from Degussa Food Ingredients GmbH) were added and stirred until a homogeneous solution was obtained. Finally, 28 g of the amino acid L-serine (component c) from Degussa Fine Chemicals) were added. The resultant dispersion was subsequently incorporated into soft gelatin capsules having a fill weight of 500 mg and a total weight of 700 mg.

Per capsule, the following were present as physiologically active composition (in percent by weight):

1% phosphatidylserine

18% docosahexaenoic acid (omega-3 fatty acid)

18% phosphatidylcholine (phospholipid)

2.8% L-serine

Molar ratios:

phosphatidylserine:phosphatidylcholine:L-serine=1:19:21

### Example 2

50 g of phosphatidylserine (Leci PS® 90PN from Degussa Food Ingredients GmbH) were added as component a) to 140 g of phosphatidylcholine (Epikuron™ 135F from Degussa Food Ingredients GmbH) as component b) and stirred in a laboratory mixer until a homogeneous mixture resulted. Subsequently, 28 g of the amino acid L-serine (component c) from Degussa Fine Chemicals) were added. The resultant dispersion was subsequently incorporated into soft gelatin capsules having a fill weight of 300 mg and a total weight of 450 mg.

Per capsule, the following were present as physiologically active composition (in percent by weight):

5% phosphatidylserine

14% phosphatidylcholine (phospholipid)

2.8% L-serine

Molar Ratios:

phosphatidylserine:phosphatidylcholine:L-serine=1:3:4

### Example 3

10 g of phosphatidylserine (Leci PS® 90PN from Degussa Food Ingredients GmbH) were added as component a) to 160 g of the omega-3 fatty acid docosahexaenoic acid (Marinol™ D-50 TG from Loders Crooklan) and stirred in a laboratory mixer until a homogeneous mixture resulted. Subsequently, 180 g of phosphatidylcholine (Epikuron™ 135F from Degussa Food Ingredients GmbH) were added as component b) and stirred until a homogeneous solution was obtained. This mixture was briefly heated to 55° C. and admixed with 50 g of beeswax as formulation aid. Finally, 28 g of the amino acid L-serine (from Degussa Fine Chemicals) were added as component c). The resultant dispersion was subsequently incorporated into soft gelatin capsules having a fill weight of 500 mg and a total weight of 700 mg.

Per capsule, the following were present as physiologically active composition (in percent by weight):

1% phosphatidylserine

16% docosahexaenoic acid (omega-3 fatty acid)

18% phosphatidylcholine (phospholipid)

2.8% L-serine

Molar Ratios:

phosphatidylserine:phosphatidylcholine:L-serine=1:19:21

### Example 4

10 g of phosphatidylserine (Leci PS® 90PN from Degussa Food Ingredients GmbH), 70 g of docosahexaenoic acid (Marinol™ DHA Powder from Loders Crooklan) as additional omega-3 fatty acid, 180 g of phosphatidylcholine (Epikuron™ 130P from Degussa Food Ingredients GmbH) as component b), and 28 g of L-serine (component c) from Degussa Fine Chemicals) were incorporated as powder mixture into hard gelatin capsules. These had a fill weight of 500 mg and a total weight of 700 mg.

Per capsule, the following were present as physiologically active composition (in percent by weight):

1% phosphatidylserine

7% docosahexaenoic acid (omega-3 fatty acid)

18% phosphatidylcholine (phospholipid)

2.8% L-serine

Molar Ratios:

phosphatidylserine:phosphatidylcholine:L-serine=1:19:21

1-12. (canceled)

13. A physiologically active composition comprising:
(a) at least one of phosphatidylserine or lysophosphatidylserine; and
(b) at least one (lyso)phospholipid different from component (a); and
(c) at least one serine source different from component (a), the components (a), (b) and (c) being present in each case in a molar ratio of a:b=1.0:0.1 to 1000, wherein b<c, and of a:c=1.0:0.1 to 1000, wherein c<b.

14. The composition of claim 13, wherein (c) is L-serine or O-phospho-L-serine.

15. The composition of claim 13, wherein the molar ratios a:b and a:c are 1.0:0.5 to 500.

16. The composition of claim 13, wherein the molar ratios a:b and a:c are 1.0:1.0 to 100.

17. The composition of claim 13, wherein under in vivo conditions, phosphatidylserine or lysophosphatidylserine can be formed from the components b) and c).

18. The composition of claim 17, wherein said phosphatidylserine or lysophosphatidylserine replaces 10 to 99% by weight of component (a) in the total formulation.

19. The composition of claim 13, wherein component (a) comprises 0.1 to 20% by weight of the total composition.

20. The composition of claim 13, wherein component (b) comprises between 15 and 65% by weight of the total composition and component (c) comprises between 0.1 and 5.0% by weight, of the total composition.

21. The composition of claim 13, further comprising an omega-3 fatty acid in amounts between 30 and 80% by weight, based on the total formulation.

22. The composition of claim 21, wherein said omega-3-fatty acid comprises between 30 and 80% by weight of the total composition.

23. The composition of claim 21, wherein said omega-3-fatty acid is docosahexaenoic acid (DHA), α-linolenic acid (ALA) or eicosapentaenoic acid (EPA), in the form of mono-, di- or triglycerides and monoesters, or mixtures thereof.

24. The composition of claim 13, further comprising at least one physiologically active constituent having circulation-promoting activity, neuroprotective or antioxidant activity, activity stimulating brain metabolism, which beneficially affects the synthesis or release of neurotransmitters, which are acetyl donors, or which beneficially affects the bioavailability, distribution and metabolism of phospholipids.

25. The composition of claim 24, wherein said constituent is selected from the group consisting of: gingko biloba, vitamin A, vitamin C, vitamin E, polyphenols, β-carotene, selenium, α-lipoic acid, complex vitamins, omega-6 fatty acids, creatine, caffeine, glucose, choline and (in-)organic salts thereof, acetylchlorine, PPAR agonists, retinoic acid receptor agonists and blood cholesterol-decreasing compounds.

26. The composition of claim 1, further comprising a formulation aid selected from the following: filler, release agent, flavoring agent and coloring agent.

27. The composition of claim 13, in a liquid or powder formulation

28. The composition of claim 27, wherein said liquid formulation is a capsule.

29. The composition of claim 27, wherein said powder formulation is a tablet or a dragee.

30. A method for improving and enhancing brain and memory function comprising administering a therapeutically effective amount of the composition of claim 13 to a subject in need thereof.

31. A method comprising administering the composition of claim 13 as dietary supplement, functional food or special nutrient to a subject in need thereof.

32. A method of treating physical and mental distress in a subject comprising administering a sufficient amount of the composition of claim 13 to a subject in need thereof.
